(19) United States
(12) Patent Application Publication
(10) Pub. No.: US 2025/0318957 A1
(43) Pub. Date: Oct. 16, 2025

(54) GOGGLE WITH INTERCHANGEABLE LENSES

(71) Applicant: Tianqi Technology Co (Ningbo) Ltd, Ningbo (CN)

(72) Inventors: James A. Chilson, Ketchum, ID (US); Roger Davis, Taipei (TW)

(21) Appl. No.: 18/632,089

(22) Filed: Apr. 10, 2024

(51) Int. Cl. A61F 9/02

(52) U.S. Cl. CPC A61F 9/025; A61F 9/026; A61F 2210/009

(57) ABSTRACT

Goggles are disclosed. As one example, a goggle includes a lens frame, at least one lens coupled to the lens frame, a first plurality of magnetic elements coupled to an inner surface of the lens frame, a goggle frame having a side portion, a second plurality of magnetic elements coupled to the goggle frame, and a mechanical clasp comprising a slider and a retention member, wherein the slider is at least partially disposed within the side portion, wherein the retention member extends from the inner surface of the lens frame, wherein the slider is configured to translate relative to the side portion between a first position and a second position, wherein the slider comprises an opening configured to receive the retention member, wherein the slider is configured to eject the retention member when the slider is translated from a first position to a second position.

### FIELD

The present disclosure relates to goggles, and in particular to goggles with interchangeable lenses.

### BACKGROUND

Users often wear goggles to protect their eyes, for example, when participating in various sporting activities, such as snow sports, motocross, mountain biking, skydiving, and paragliding. Conventional goggles for sporting activities are designed with a frame and a lens that is fixedly mounted to the frame, requiring users to own multiple pairs of goggles for different use conditions. To address this concern, some goggles for sporting activities are designed with interchangeable lenses that are releasably coupled to a frame, enabling users to exchange lenses and adapt the same goggle for different use conditions. The frame of such goggles may be equipped with an attachment mechanism to releasably attach the lens to the frame. However, removal of the lens in existing such goggles may be overly complex or cumbersome for a user, or may have other deficiencies that may result in a less than ideal user experience. For example, in some goggles, the lens can be attached to the frame with magnets, resulting in a lens that may be easily dislodged from the frame.

### SUMMARY

Described herein are embodiments of a goggle having a magnetic and mechanical attachment mechanism that addresses some of the drawbacks in the prior art.

According to some implementations, a goggle comprises a lens frame, at least one lens coupled to the lens frame, a first plurality of magnetic elements coupled to an inner surface of the lens frame, a goggle frame having a side portion, a second plurality of magnetic elements coupled to the goggle frame, and a mechanical clasp comprising a slider and a retention member. The slider can be at least partially disposed within the side portion. The retention member can extend from the inner surface of the lens frame. The slider can be configured to translate relative to the side portion between a first position and a second position. The slider can comprise an opening configured to receive the retention member. The slider can be configured to eject the retention member when the slider is translated from the first position to the second position.

The mechanical clasp can further comprise a spring configured to bias the slider towards the first position.

The slider can have a first end portion and a second end portion, wherein the first end portion extends out of the goggle frame, and wherein the second end portion is coupled to the spring.

A channel can be disposed within the side portion of the goggle frame. The slider can be at least partially disposed within the channel.

The retention member can comprise a hook having a first angled surface configured to contact a first edge of the opening when the lens frame is coupled to the goggle frame.

The hook can include a second angled surface configured to contact a second edge of the opening when the lens frame is removed from the goggle frame.

The goggle frame can comprise a face gasket and a goggle frame insert coupled to the face gasket, wherein the goggle frame insert at least partially defines the side portion of the goggle frame.

The slider can be at least partially disposed within the goggle frame insert.

The goggle frame insert can include a window. The window and the opening of the slider can be axially offset in the first position, and the window and the opening of the slider can be axially aligned in the second position.

The slider can be configured to translate along a length of the side portion.

According to some implementations, a goggle comprises a lens assembly comprising a lens frame and at least one lens coupled to the lens frame, wherein the lens frame comprises a projection extending from an inner surface of the lens frame, a first plurality of magnetic elements coupled to the inner surface of the lens frame, a goggle frame comprising a face gasket and a goggle frame insert, a second plurality of magnetic elements coupled to the face gasket and the goggle frame insert, and a slider at least partially disposed within the goggle frame insert, the slider comprising an opening configured to receive and retain the projection, wherein the slider is configured to translate relative to the goggle frame insert between a first position and a second position, wherein the slider applies a force against the projection to eject the lens assembly when the slider is translated to the second position.

The slider can be spring-biased towards the first position.

The goggle frame insert can at least partially defines a side portion of the goggle frame.

The face gasket can comprise thermoplastic polyurethane (TPU).

The projection can comprise an angled entry surface and an angled eject surface.

The slider can apply the force against the angled eject surface to eject the lens assembly when the slider is translated to the second position.

According to some implementations, the goggle comprises a lens assembly comprising a lens frame and at least one lens coupled to the lens frame, a goggle frame configured for placement around eyes of a user, and an attachment mechanism configured to couple the lens assembly to the goggle frame. The attachment mechanism comprises a plurality of magnetic elements and mechanical elements. The magnetic elements are coupled to the lens frame and the goggle frame. The mechanical elements comprise a slider coupled to the goggle frame and a retention member extending from an inner surface of the lens frame and configured to releasably couple with an opening of the slider. The slider is configured to translate relative to the goggle frame between a first position and a second position. The slider applies a force against the retention member to eject the lens assembly when the slider is translated to the second position.

The retention member can comprise an angled entry ramp and an angled eject ramp. The slider applies the force against the angled eject ramp to eject the lens assembly when the slider is translated to the second position.

The goggle frame comprises a face gasket and a pair of goggle frame inserts coupled to the face gasket at either side of the goggle frame. The slider can be at least partially disposed within the goggle frame insert.

At least one magnetic element of the plurality of magnetic elements cam be coupled to the goggle frame insert.

An end portion of the slider can extend beyond a lower portion of the goggle frame. The end portion can comprise a flange.

According to some implementations, a goggle comprises a lens assembly comprising a lens frame having an outer surface and an inner surface, a first lens coupled to the outer surface of the lens frame, and a second lens coupled to the inner surface of the lens frame. The lens frame can include an upper portion, a lower portion, two side portions extending between the upper portion and the lower portion, and a nose-accommodating portion. Each side portion of the lens frame can comprise a projection extending from the inner surface of the lens frame. The goggle also comprises a first plurality of magnets coupled to the inner surface of the lens frame, wherein the first plurality of magnets are coupled to the upper portion, the side portions, and the nose-accommodating portion of the lens frame. The goggle also comprises a goggle frame having an upper portion, a lower portion, two side portions extending between the upper portion and the lower portion, and a nose-accommodating portion. The goggle frame can comprise a face gasket and two goggle frame inserts coupled to the face gasket. The two goggle frame inserts can at least partially define the two side portions of the goggle frame. The goggle also comprises a second plurality of magnets coupled to the face gasket and the two goggle frame inserts. The second plurality of magnets are coupled to the upper portion, the side portions, and the nose-accommodating portion of the goggle frame. The first plurality of magnets and the second plurality of magnets are configured to releasably couple together. The goggle also comprises a spring-biased slider at least partially disposed within each goggle frame insert. The slider can comprise an opening configured to receive one of the projections to releasably couple the lens assembly to the goggle frame.

### BRIEF DESCRIPTION OF THE DRAWINGS

FIG. 1 is a perspective view of a goggle, according to one example.

FIG. 2 is a plan view of a lens assembly of the goggle of FIG. 1.

FIG. 3 is a plan view of a lens frame of the goggle of FIG. 1.

FIG. 4 is a plan view of a goggle frame of the goggle of FIG. 1.

FIG. 5 is a detailed view of a side portion of the goggle frame of FIG. 4.

FIG. 6 is a front plan view of a goggle frame insert of the goggle of FIG. 1.

FIG. 7 is a rear plan view of the goggle frame insert of FIG. 6.

FIG. 8 is a detailed view of a side portion of the lens frame of FIG. 3.

FIG. 9 is a partial perspective view of the lens frame of FIG. 3 and the goggle frame insert of FIG. 6 in an unattached configuration.

FIG. 10 is a perspective view of the goggle of FIG. 1.

FIG. 11 is a front view of the goggle of FIG. 1.

FIG. 12 is a top view of the goggle of FIG. 1.

FIGS. 13 and 14 are side views of the goggle of FIG. 1.

FIG. 15 is a bottom view of the goggle of FIG. 1.

FIG. 16 is a rear view of the goggle of FIG. 1.

### DETAILED DESCRIPTION

### General Considerations

Described below are implementations of goggles with an attachment mechanism that enables simple and convenient exchange of a lens assembly on a goggle frame, for example, to enable a user to easily change the lens assembly used with the goggle frame. In some implementations, the attachment mechanism includes a first retainer, such as magnetic elements, and/or a second retainer, such as mechanical elements, to enable such releasable attachment. In some examples, the first retainer and the second retainer are operable exclusive of each other. For example, the goggles in some implementations can include magnets disposed around both the lens assembly and the goggle frame as well as mechanical elements to releasably couple the lens assembly and the goggle frame.

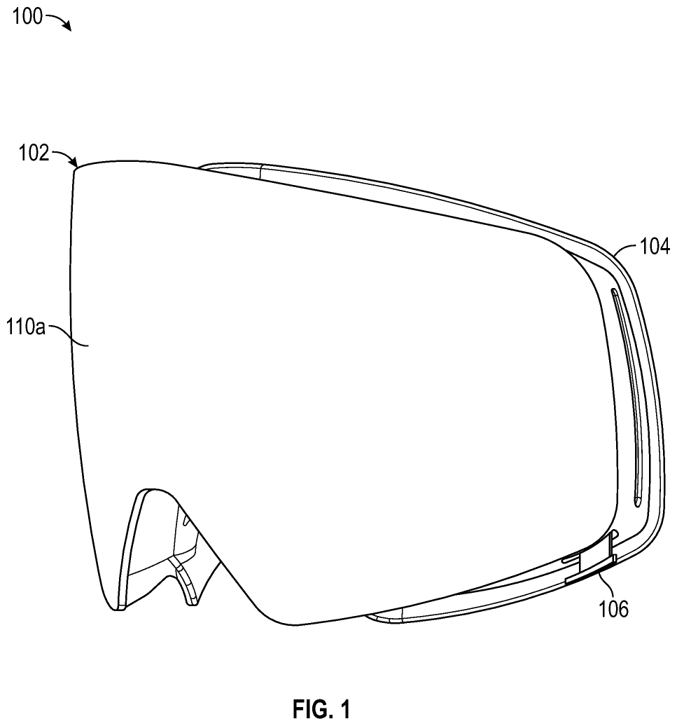

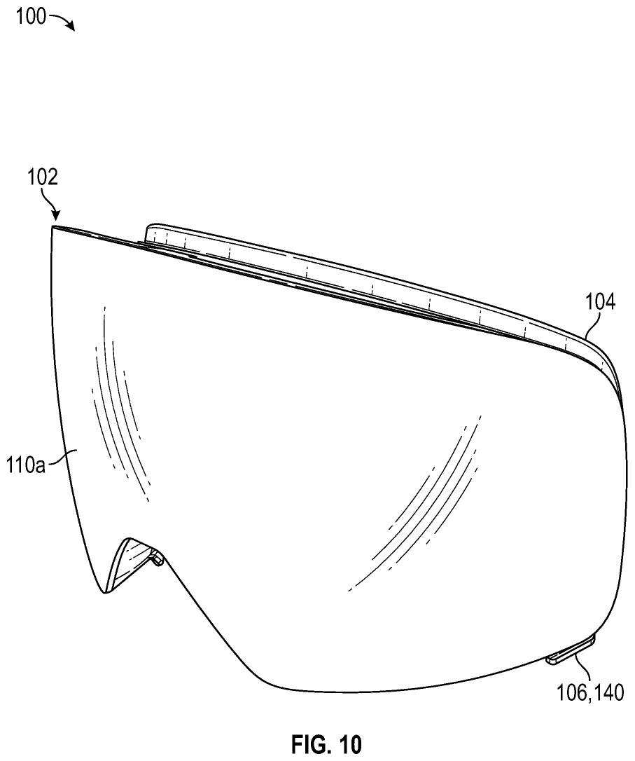

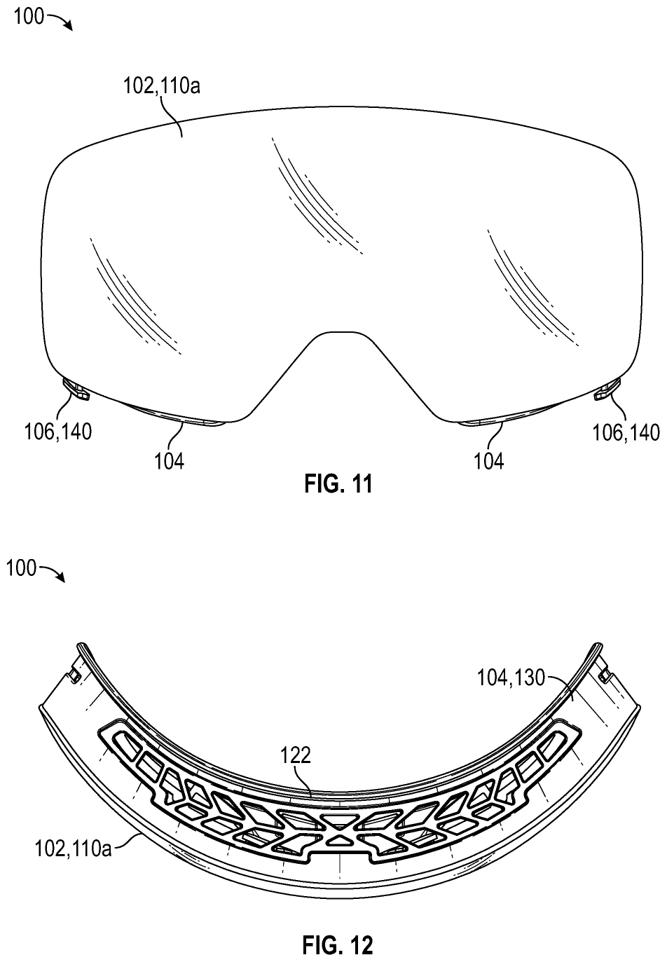

Referring to FIG. 1, an exemplary goggle 100 is shown having a lens assembly 102 that is releasably coupled to a goggle frame 104. The goggle frame 104 is configured for placement on a face of a user, for example, around the eyes of the user. The lens assembly 102 is releasably coupled to the goggle frame 104 by an attachment mechanism 106 (see also FIGS. 10-11). The attachment mechanism 106 allows the user to exchange the lens assembly 102 that is attached to the goggle frame 104, for example, to adjust to different use conditions. For example, lens assemblies can have different tints such as clear, yellow, polarized, etc. and be suitable for different use conditions such as full sun conditions, low light conditions, and the like.

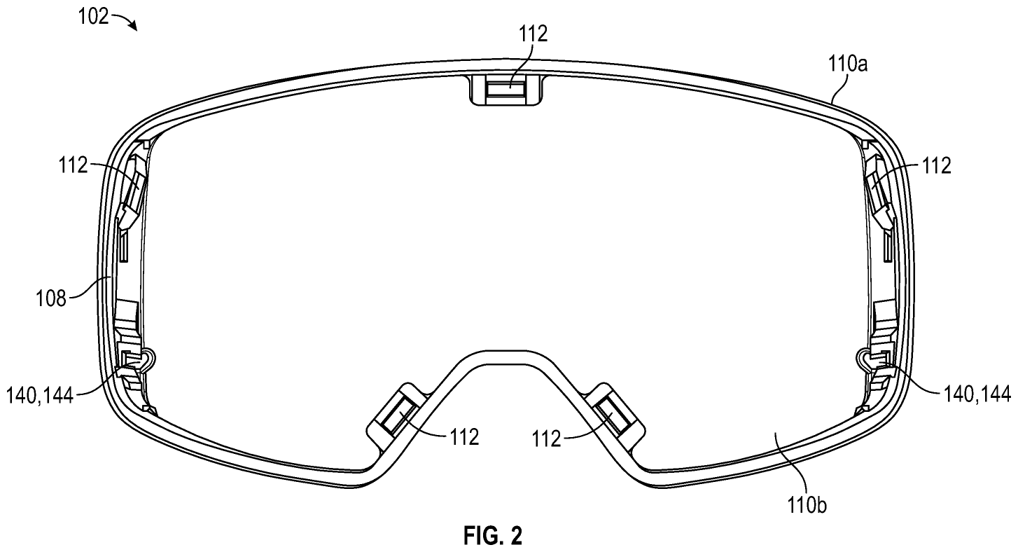

The lens assembly 102 includes at least one lens coupled to a lens frame 108, as shown in FIG. 2. In some examples, the lens assembly 102 can include multiple lenses, such as an outer lens 110a coupled to an outer surface of the lens frame 108 and an inner lens 110b coupled to an inner surface of the lens frame 108. In some examples, as shown, the outer lens 110a can extend outwards beyond an outer edge of the lens frame 108.

The attachment mechanism 106 includes a plurality of magnetic elements 112 configured to magnetically couple the lens assembly 102 to the goggle frame 104. Magnetic elements 112 are disposed around the perimeter of the lens frame 108 and around the perimeter of the goggle frame 104. The magnetic elements 112 coupled to the lens frame 108 are aligned with the magnetic elements 112 coupled to the goggle frame 104, such that the magnetic elements 112 magnetically couple the lens assembly 102 to the goggle frame 110 when the lens assembly 102 is aligned with and positioned adjacent to the goggle frame 110.

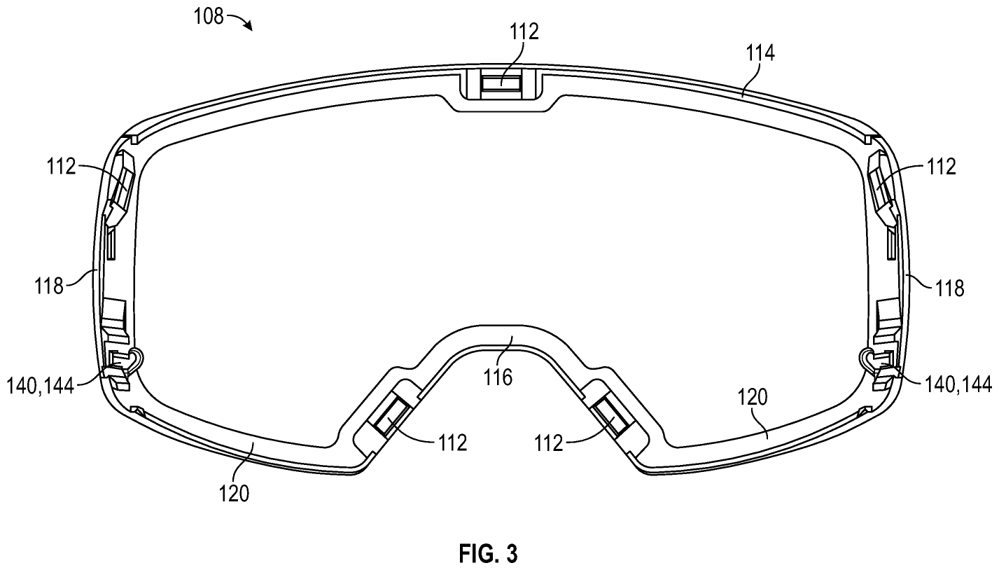

In some examples, as shown in FIGS. 2 and 3, the attachment mechanism 106 includes five pairs of magnetic elements 112. In other examples, a greater or lesser number of pairs of magnetic elements 112 can be used. In the illustrated example, with reference to lens frame 108, a first magnetic element 112 is coupled to an upper portion 114 of the lens frame 108 and is aligned with a nose-accommodating portion 116 of the lens frame 108. Second and third magnetic elements 112 are coupled to side portions 118 of the lens frame 108 at a location adjacent to the upper portion 114. Fourth and fifth magnetic elements 112 are coupled to opposite sides of the nose-accommodating portion 116 of the lens frame 108 adjacent to a lower portion 120 of the lens frame 108.

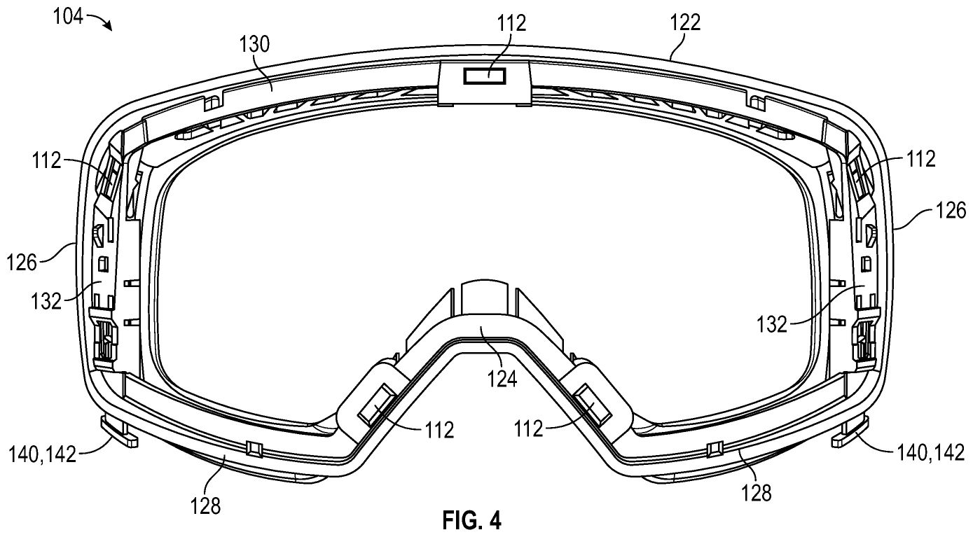

The magnetic elements 112 coupled to the goggle frame 104 are disposed at similar locations around the goggle frame 104 as those described above in connection with the lens frame 108. For example, as shown in FIG. 4, a first magnetic element 112 is coupled to an upper portion 122 of the goggle frame 104 and is aligned with a nose-accommodating portion 124 of the goggle frame 104. Second and third magnetic elements 112 are coupled to side portions 126 of the goggle frame 104 at a location adjacent to the upper portion 122. Fourth and fifth magnetic elements 112 are coupled to opposite sides of the nose-accommodating portion 124 of the goggle frame 104 adjacent to a lower portion 128 of the goggle frame 104. It should be appreciated that, in some examples, the same or different number of magnetic elements 112 can be disposed at different locations on the lens frame 108 and/or goggle frame 104.

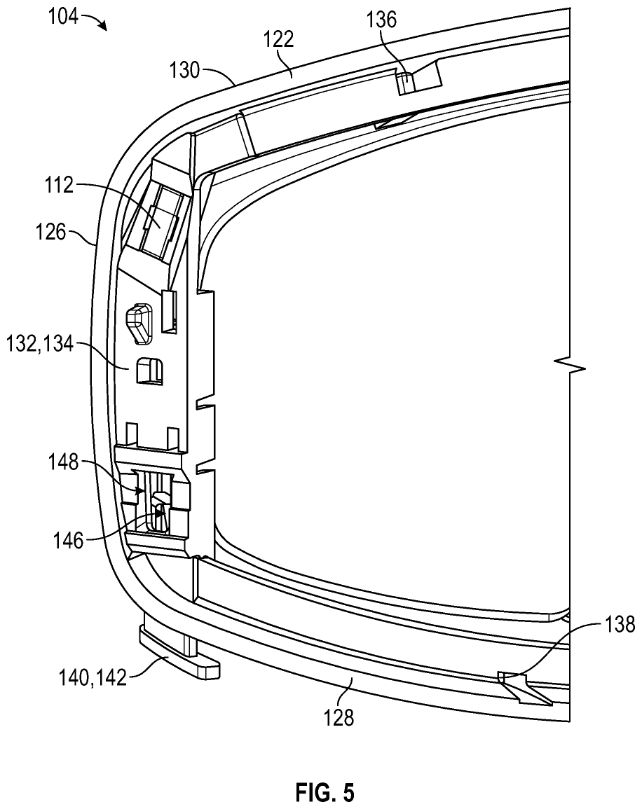

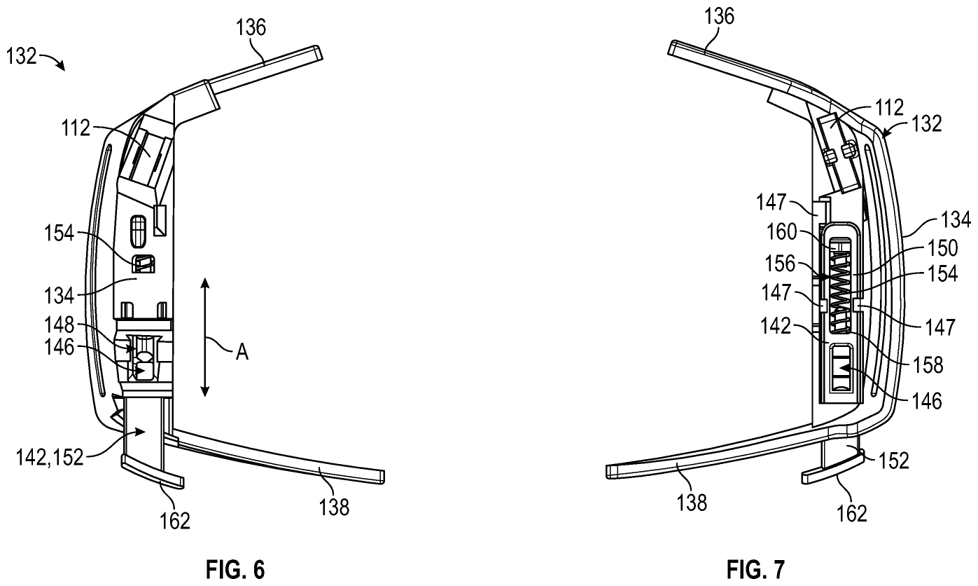

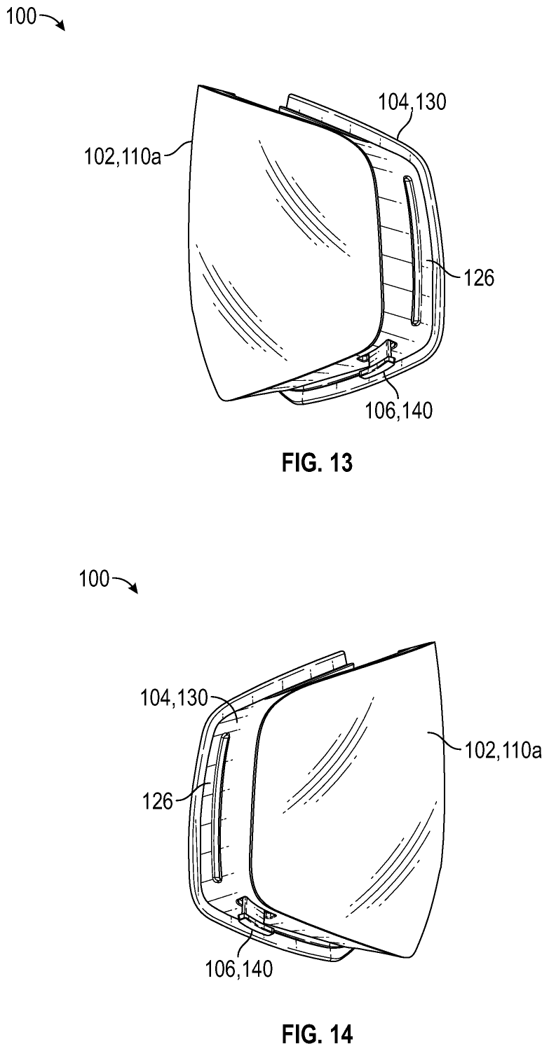

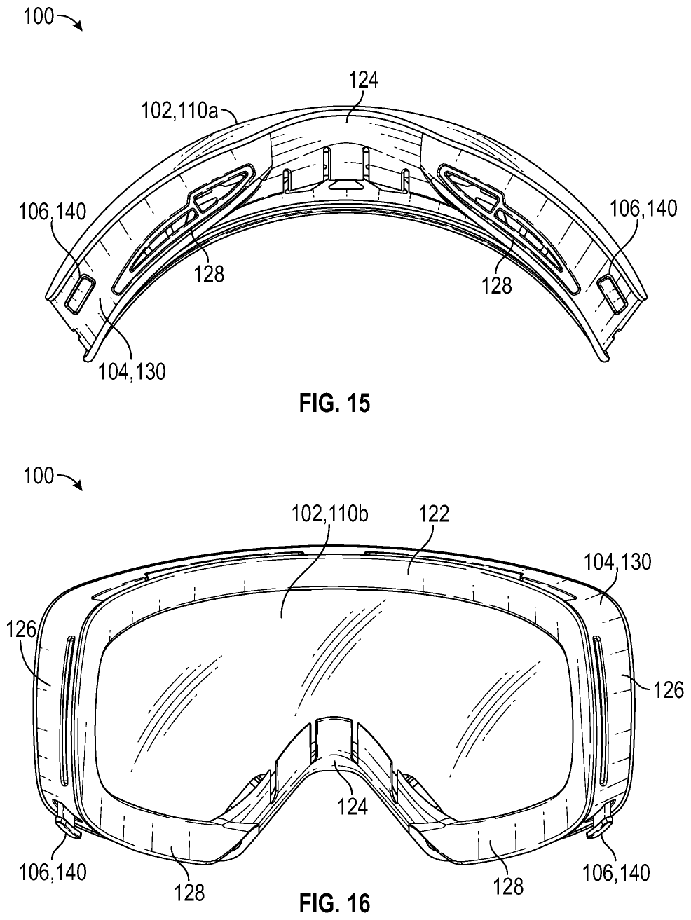

In some examples, the goggle frame 104 includes a face gasket 130 (FIGS. 4 and 12-16) extending around a perimeter of the goggle frame 104 and a pair of goggle frame inserts 132 coupled to the face gasket 130 on either side of the goggle frame 104. Each goggle frame insert 132 at least partially defines the side portion 126 of the goggle frame 104, which is shown in greater detail in FIG. 5. The goggle frame insert 132 comprises a main body 134 as well as a first extension portion 136 and a second extension portion 138 that extend from the main body 134 (see also FIGS. 6 and 7). The first extension portion 136 is coupled to the face gasket 130 within the upper portion 122 of the goggle frame 104 and the second extension portion 138 is coupled to the face gasket 130 within the lower portion 128 of the goggle frame 104. In some examples, the face gasket 130 comprises a flexible material, such as thermoplastic polyurethane (TPU). In some examples, as shown, the face gasket 130 and the goggle frame insert 132 are separate and distinct components. In other examples, the face gasket 130 and the goggle frame insert 132 can form a single, unitary component.

In addition to magnetic elements 112, the attachment mechanism 106 can also include a pair of mechanical elements 140 on either side of the goggle 100 (see also FIGS. 10-16). In some examples, the mechanical elements 140 are also referred to herein as mechanical clasps. The mechanical elements 140 can be configured to provide supplemental retention in addition to the magnetic elements 112. In some examples, the mechanical elements 140 can operate independent of the magnetic elements 112, such that each of the magnetic elements 112 and the mechanical elements 140 can releasably couple the lens assembly 102 to the goggle frame 104. In some examples, the attachment mechanism 106 can include mechanical elements 140 in lieu of magnetic elements 112.

Each mechanical element 140 includes a slider 142 that is coupled to the goggle frame 104 and a retaining member 144 that is coupled to the lens frame 108. As described in more detail below, the slider 142 and the retaining member 144 are configured to releasably engage with one another, for example, to releasably couple the lens assembly 102 to the goggle frame 104. Specifically, in some examples, the retaining member 144 can be configured to pass through an opening 146 of the slider 142 and engage with at least one surface of the slider 142 to releasably couple the lens assembly 102 to the goggle frame 104.

Each slider 142 is positioned at least partially within one of the side portions 126 of the goggle frame 104. In some examples, as shown in FIGS. 5-7, the slider 142 is at least partially disposed within the goggle frame insert 132. The slider 142 can be configured to translate relative to the goggle frame insert 132 in direction A, along a length of the goggle frame insert 132 (e.g., along an axis extending from the upper portion 122 to the lower portion 128 of the goggle frame 104). In some examples, the goggle frame insert 132 includes a plurality of tabs 147 which partially define a channel or track in which the slider 142 is configured to translate.

The goggle frame insert 132 includes a window 148 that is at least partially aligned with the opening 146 of the slider 142. The window 148 provides an opening through the goggle frame insert 132 for the retention member 144 to access and engage with the opening 146 of the slider 142, for example, such that the retention member 144 can be coupled to the slider 142. In some examples, the slider 142 is configured to translate from a first position, shown in FIGS. 5-7, in which the opening 146 and the window 148 are offset in direction A to a second position in which the opening 146 is aligned with the window 148 and fully accessible through the window 148. Specifically, in the first position, an upper edge of the opening 146 can be axially between an upper edge of the window 148 and a lower edge of the window 148 and a lower edge of the opening 146 can be below the lower edge of the window 148, such that only a portion of the opening 146 is accessible through the window 148 in the first position.

The slider 142 includes a first end portion 150 disposed within the goggle frame insert 132 and a second end portion 152 that extends beyond the goggle frame insert 132 and beyond the lower portion 128 of the goggle frame 104, such that the second end portion 152 is accessible to a user wearing the goggle 100. The first end portion 150 can be coupled to a biasing element 154 (e.g., a spring) within the goggle frame insert 132. The biasing element 154 can bias the slider 142 in a downwards direction, for example, away from the upper portion 122 of the goggle frame 104 and towards the lower portion 128 of the goggle frame 104. In the first position, shown in FIGS. 5-7, the biasing element 154 biases the slider 142 downwards, such that the opening 146 is axially offset from the window 148 (e.g., a lower edge of the opening 146 is below a lower edge of the window 148). In some examples, as shown in FIG. 7, the first end portion 150 can comprise an opening 156 in which the biasing element 154 is disposed. For example, a first end of the biasing element 154 can be coupled to a lower end 158 of the opening 156 and a second end of the biasing element 154 can be coupled to a projection 160 of the goggle frame insert 132 that extends through the opening 156. In this way, as the slider 132 is moved in an upwards direction (e.g., when a user pushes the second end portion 152), the lower end 158 of the opening 156 translates towards the projection 160, causing the biasing element 154 to compress.

In some examples, the second end portion 152 of the slider 142 comprises a user-engagement portion 162, such as a flange, to facilitate easier manipulation of the slider 142. For example, a user can press the user-engagement portion 162 to move the slider 142 within the goggle frame insert 132, for example, in an upwards direction. In some examples, the user-engagement portion 162 is sized to prevent the second end portion 152 of the slider 142 from translating entirely within the side portion 126, such that the user-engagement portion 162 remains outside of the goggle frame 104 during use.

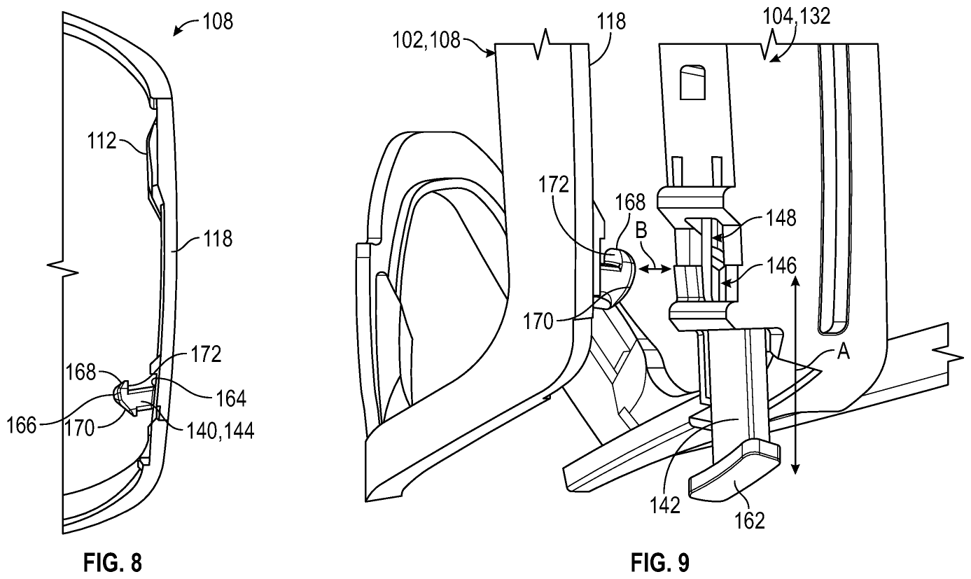

As introduced above, the retaining member 144 can pass through the opening 146 of the slider 142 to couple the lens assembly 102 to the goggle frame 104. As shown in FIG. 8, the retention member 144 can comprise a hook or latch that is configured to engage with the slider 142. In some examples, as shown, the retention member 144 projects outwardly and away from the side portion 118 of the lens frame 108. The retention member 144 extends outwardly at an angle, for example, in a direction substantially perpendicular to a length of the side portion 118 of the lens frame 108.

The retention member 144 has a first end 164 coupled to the lens frame 108 and a second end 166 extending outwards from the lens frame 108 (e.g., towards the goggle frame 104). The second end 166 of the retention member 144 is defined by a first angled surface 168 and a second angled surface 170. The first angled surface 168 can facilitate easier entry of the retention member 144 through the opening 146 of the slider 142 and is also referred to herein as an entry ramp. The second angled surface 170 can facilitate easier ejection of the retention member 144 from the opening 146 of the slider 142 and is also referred to herein as an eject ramp. The retention member 144 can also comprise a vertical surface 172 that is configured to hook or catch onto a portion of the slider 142, for example, a surface adjacent to the opening 146 of the slider 142.

To engage the retaining member 144 with the slider 142, the lens assembly 102 can be brought into alignment with the goggle frame 104, for example, such that the magnetic elements 112 of the lens frame 108 are aligned with the magnetic elements 112 of the goggle frame 104. Once the magnetic elements 112 are aligned, the retaining member 144 is partially aligned with the opening 146 of the slider 142. For example, in the first position, the opening 146 of the slider 142 can be slightly below the retaining member 144, such that the first angled surface 168 is aligned with an upper edge of the opening 146. In some examples, the upper edge of the opening 146 of the slider 142 is angled or beveled to match the first angled surface 168.

As shown in FIG. 9, the user can then move the lens frame 108 in direction B, bringing the lens assembly 102 towards the goggle frame 104. The first angled surface 168 of the retaining member 144 can push against the upper edge of the opening 146 of the slider 142, which in turn causes the slider 142 to translate upwards towards the upper portion 122 of the goggle frame 104. This enables the retaining member 144 to pass through the opening 146 of the slider 142. Once the retaining member 144 is through the opening 146, the biasing member 140 can cause the slider 142 to translate downwards towards the lower surface 126 of the goggle frame 104, such that at least one surface of the opening 146 engages with the retaining member 144, for example, the vertical surface 172 against an inner surface of the slider 142.

To remove the lens assembly 102 from the goggle frame 104, the retaining member 144 is released from the slider 142. Specifically, a user can actuate the slider 142 by pressing the second end portion 152 of the slider 142 in an upwards direction (e.g., towards the upper portion 122 of the goggle frame 104). A lower edge of the opening 146 of the slider 142 can press against the second angled surface 170 of the retaining member 144 as the slider 142 translates upwards. The contact between the lower edge of the opening 146 and the second angled surface 170 causes the retaining member 144 to move away from the goggle frame 104 in direction B, ejecting the retaining member 144 from the opening 146 of the slider 142. The lens assembly 102 can then be removed from the goggle frame 104 by uncoupling the magnetic elements 112.

The attachment mechanism 106 includes magnetic elements 112 and/or mechanical elements 140 to provide a secure and convenient releasable attachment between the lens assembly 102 and the goggle frame 104. For example, the mechanical elements 140 can prevent inadvertent detachment of the lens assembly from the magnetic elements 112. The lens assembly 102 can be removed from the goggle frame 104 in a simplified manner, for example, by simply pressing the user-engagement portion 162 of the slider 142 to uncouple the mechanical elements 140 and detaching the lens assembly 102 from the magnetic elements 112. In this way, the user need only push the slider 142 to disengage the lens assembly 102 and can engage the lens assembly 102 by pushing the retaining member 144 into the opening 146 of the slider 142 and overcoming the spring force. In some examples, the attachment mechanism 106 enables the lens assembly 102 to be attached to and removed from the goggle frame 104 while a user is wearing the goggle 100, for example, while a user rides a chair lift, etc.

The illustrated goggles are sports goggles, i.e., snow goggles, but the same principles can be applied to protective goggles for other sports such as, motocross, downhill biking, skydiving, paragliding, and other such activities.

The features described herein with regard to any example can be combined with other features described in any one or more of the other examples, unless otherwise stated. For example, any one or more of the features of one goggle can be combined with any one or more features of another goggle. As another example, any one or more features of one attachment mechanism can be combined with any one or more features of another attachment mechanism.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

The invention claimed is:

1. A goggle comprising:
a lens frame;
at least one lens coupled to the lens frame;
a first plurality of magnetic elements coupled to an inner surface of the lens frame;
a goggle frame having a side portion with a length between a lower portion and an upper portion;
a second plurality of magnetic elements coupled to the goggle frame; and
a mechanical clasp comprising a slider and a retention member, wherein the slider is at least partially disposed within the side portion, wherein the retention member extends from the inner surface of the lens frame, wherein the slider is configured to translate along the length of the side portion between a first position and a second position, wherein the slider comprises an opening configured to receive the retention member, wherein the slider is configured to eject the retention member when the slider is translated from the first position to the second position.

2. The goggle of claim 1, wherein the mechanical clasp further comprises a spring configured to bias the slider towards the first position.

3. The goggle of claim 2, wherein the slider has a first end portion and a second end portion, wherein the first end portion extends out of the goggle frame, and wherein the second end portion is coupled to the spring.

4. The goggle of claim 1, further comprising a channel disposed within the side portion of the goggle frame, wherein the slider is at least partially disposed within the channel.

5. The goggle of claim 1, wherein the retention member comprises a hook having a first angled surface configured to contact a first edge of the opening when the lens frame is coupled to the goggle frame.

6. The goggle of claim 5, wherein the hook includes a second angled surface configured to contact a second edge of the opening when the lens frame is removed from the goggle frame.

7. The goggle of claim 1, wherein the goggle frame comprises a face gasket and a goggle frame insert coupled to the face gasket, wherein the goggle frame insert at least partially defines the side portion of the goggle frame.

8. The goggle of claim 7, wherein the slider is at least partially disposed within the goggle frame insert.

9. The goggle of claim 7, wherein the goggle frame insert comprises a window, wherein the window and the opening of the slider are axially offset in the first position, and wherein the window and the opening of the slider are axially aligned in the second position.

10. (canceled)

11. A goggle comprising:
a lens assembly comprising a lens frame and at least one lens coupled to the lens frame, wherein the lens frame comprises a projection extending from an inner surface of the lens frame;
a first plurality of magnetic elements coupled to the inner surface of the lens frame;
a goggle frame comprising a face gasket and a goggle frame insert extending between upper and lower portions of the goggle frame;
a second plurality of magnetic elements coupled to the face gasket and the goggle frame insert; and
a slider at least partially disposed within the goggle frame insert, the slider comprising an opening configured to receive and retain the projection, wherein the slider is configured to translate relative to the goggle frame insert along a direction between the lower and upward portions between a first position and a second position, wherein the slider applies a force against the projection to eject the lens assembly when the slider is translated to the second position.

12. The goggle of claim 11, wherein the slider is spring-biased towards the first position.

13. The goggle of claim 11, wherein the goggle frame insert at least partially defines a side portion of the goggle frame.

14. The goggle of claim 11, wherein the face gasket comprises thermoplastic polyurethane (TPU).

15. The goggle of claim 11, wherein the projection comprises an angled entry surface and an angled eject surface.

16. The goggle of claim 15, wherein the slider applies the force against the angled eject surface to eject the lens assembly when the slider is translated to the second position.

17. A goggle comprising:
a lens assembly comprising a lens frame and at least one lens coupled to the lens frame;
a goggle frame configured for placement around eyes of a user and having a lower portion and an upper portion; and
an attachment mechanism configured to couple the lens assembly to the goggle frame, wherein the attachment mechanism comprises a plurality of magnetic elements and mechanical elements, wherein the magnetic elements are coupled to the lens frame and the goggle frame, wherein the mechanical elements comprise a slider coupled to the goggle frame and a retention member extending from an inner surface of the lens frame and configured to releasably couple with an opening of the slider, wherein the slider is configured to translate relative to the goggle frame along a direction between the lower portion and the upper portion between a first position and a second position, and wherein the slider applies a force against the retention member to eject the lens assembly when the slider is translated to the second position.

18. The goggle of claim 17, wherein the retention member comprises an angled entry ramp and an angled eject ramp, and wherein the slider applies the force against the angled eject ramp to eject the lens assembly when the slider is translated to the second position.

19. The goggle of claim 17, wherein the goggle frame comprises a face gasket and a pair of goggle frame inserts coupled to the face gasket at either side of the goggle frame, wherein the slider is at least partially disposed within the goggle frame insert.

20. The goggle of claim 19, wherein at least one magnetic element of the plurality of magnetic elements is coupled to the goggle frame insert.

21. The goggle of claim 17, wherein an end portion of the slider extends beyond the lower portion of the goggle frame, wherein the end portion comprises a flange.

22. A goggle comprising:
a lens assembly comprising a lens frame having an outer surface and an inner surface, a first lens coupled to the outer surface of the lens frame, and a second lens coupled to the inner surface of the lens frame, wherein the lens frame includes an upper portion, a lower portion, two side portions extending between the upper portion and the lower portion, and a nose-accommodating portion, wherein each side portion of the lens frame comprises a projection extending from the inner surface of the lens frame;
a first plurality of magnets coupled to the inner surface of the lens frame, wherein the first plurality of magnets are coupled to the upper portion, the side portions, and the nose-accommodating portion of the lens frame;
a goggle frame having an upper portion, a lower portion, two side portions extending between the upper portion and the lower portion, and a nose-accommodating portion, the goggle frame comprising a face gasket and two goggle frame inserts coupled to the face gasket, wherein the two goggle frame inserts at least partially define the two side portions of the goggle frame;
a second plurality of magnets coupled to the face gasket and the two goggle frame inserts, wherein the second plurality of magnets are coupled to the upper portion, the side portions, and the nose-accommodating portion of the goggle frame, wherein the first plurality of magnets and the second plurality of magnets are configured to releasably couple together; and
a spring-biased slider at least partially disposed within each goggle frame insert, the slider comprising an opening configured to receive one of the projections to releasably couple the lens assembly to the goggle frame, wherein the slider is configured to translate along a direction between the lower portion and the upper portion between a first position and a second position, and wherein the slider is configured to eject the lens assembly when the slider is translated to the second position.
